(19) United States
(12) Patent Application Publication
(10) Pub. No.: US 2024/0304306 A1
(43) Pub. Date: Sep. 12, 2024

(54) COMPUTER USER INTERFACE MODIFICATION BASED ON BIOMETRIC DATA

(71) Applicant: Lenovo (Singapore) Pte. Ltd., Singapore (SG)

(72) Inventors: Leana Neparidze, San Fransisco, CA (US); Matthew Fardig, Boonville, IN (US); Lindsay Nelson, Highlands Ranch, CO (US); Inna Zolin, Cary, NC (US)

(21) Appl. No.: 18/179,853

(22) Filed: Mar. 7, 2023

(51) Int. Cl. G16H 20/70; G06F 3/01

(52) U.S. Cl. CPC G16H 20/70; G06F 3/015

(57) ABSTRACT

A system and process display a computer user interface to a person, monitor biometric data of the person during the display of the computer user interface, and modify the computer user interface as a function of the biometric data.

### TECHNICAL FIELD

Embodiments described herein generally relate to modifying computer user interfaces, and in an embodiment, but not by way of limitation, modifying computer user interfaces based on biometric data.

### BACKGROUND

Virtually everyone uses computers and the Internet nowadays in connection with some aspect of daily life. This use can involve such things as computer-based tests, chat sessions, online browsing of websites, online job applications, online banking sessions, and online shopping sessions. These computer uses provide many benefits to people. For example, computer-based testing allows for test takers to have the same testing experience whether they are in a classroom or at home using their own computer. Testing of this nature is used in almost every major industry, as well as in school classrooms.

A particular computer-based testing, known as Computerized Adaptive Testing (CAT), is a common approach used in testing situations as it allows for more efficient testing, and testing that adapts to the test taker's ability. It is often referred to as “tailored testing.” The test itself adapts on the fly based on the correctness of the answers to previous questions. For example, if a test taker correctly answers a few questions in a given area, the test assumes that the test taker has knowledge in that area and moves to other topics for testing. If a test taker incorrectly answers questions in a certain area, a test can end early, or the test can continue to adapt by asking more questions in that area.

Consequently, tests with static sets of questions are often replaced by adaptive tests. These tests start with a mid-level knowledge question. The question is presented to the test taker, who then answers it, either correctly or incorrectly. Based on that answer (and other answers), the test selects another question, either in the same topic or a different topic as the previous question. Testing continues until some sort of termination threshold is reached (e.g., the test taker demonstrates acceptable knowledge in all test areas, the test taker was unable to answer enough questions in a given area, etc.). With CAT, different test takers might receive an entirely different test taking experience for the same test.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings.

FIG. 1 is a block diagram illustrating operations and features of a system and process to modify a computer user interface based on biometric data.

FIG. 2 is a block diagram of a computer architecture upon which one or more of the embodiments disclosed herein can execute.

### DETAILED DESCRIPTION

The present disclosure relates to modifying a computer user interface based on biometric data, and in particular, biometric data that are collected from a user of the computer user interface. In an embodiment, the computer user interface relates to a computer-based test. However, the computer-based test is only one example, and the present disclosure is not limited to computer-based testing.

One type of computer-based testing is known as Computerized Adaptive Testing (CAT). CAT, and test taking in general, does not consider the test taker himself or herself. The tests are either predesigned to flow in a certain order or adapt based on the correctness of previous answers. These CAT tests do not consider one or more of the following issues. The test taker might get nervous during the test. This nervousness can cause the test taker to incorrectly answer some questions. The test taker might miss a question (and know that they missed it) and may not be able to recover from any resulting negative emotions (such as fear of failure) that arise in this situation. This can cause the remainder of the test to continue in a negative direction and not accurately reflect the test taker's knowledge.

There are proven techniques however that can help test takers relax and perform better in test taking. For example, simple breathing exercises or techniques to clear a test taker's mind can be used to calm down the test taker. But testing often does not allow sufficient time for these techniques to be used effectively.

Also, timed tests can lead to situations where a test taker hurries through the test due to the fear of not getting to every question. This can result in the test taker making unexpected mistakes. Additionally, external distractions can occur during test taking. When a test taker is distracted, they will not perform to their optimal level.

In view of these issues, an embodiment of the present disclosure harvests biometric data from test takers. These biometric data are used to determine what is going on with the test taker and to allow the testing process to adapt based on these biometric data. A goal is to ensure that a test taker has an optimal test taking experience, and that a test taker's knowledge is actually and fairly evaluated, rather than a test taker's ability to take a test effectively.

The biometric data can be sensed in a variety of manners. For example, smart watches can gather information such as oxygen levels, breathing rate, heart rate and blood pressure. Cameras can be used to monitor the test taker's eyes, and thereby determine the attention level of the test taker. Also, the dilation of a test taker's eyes may reveal some information regarding the state of the test taker. In more specialized setups, an electroencephalogram (EEG) can be used to determine the brain activity of the test taker, and the test can be adapted accordingly. Specifically, in one or more embodiments, the test can be adapted in one of the several following ways.

A particular question may cause the test taker's breathing to increase, thereby indicating that the test taker is nervous. In an embodiment, the test can adapt by presenting an easy question to the test taker in order to calm the test taker. Or the test can present a question to the test taker that the test taker previously answered correctly, but the question is presented in a slightly different wording or format. Or the test can present for review a question that the test taker previously answered correctly (and perhaps inform the test taker that they answered the question correctly). These positive reinforcement techniques allow the test taker to calm down and gain some confidence before the next question is presented to the test taker.

A particular question may be very lengthy, and this may cause the test taker's blood pressure to rise. The testing process may adapt by offering shorter questions.

A particular question may require a user to choose several correct answers, and the biometric data may indicate that the test taker's anxiety level has gone up. In response to this, the testing process can adapt by breaking the question up into multiple questions that only require a single correct answer. That is, the style of the question can be adapted.

As another example, the biometric data may determine that a test taker's stress or anxiety level has been high for a certain number of question and/or a certain period of time. The testing process can then pause itself, and the testing process can suggest that the user partake in a relaxation technique, such as a breathing exercise, before the test taker is presented with the next question. In an embodiment, the test time can be automatically increased to allow for this relaxation technique so that the test taker does not lose any test taking time.

In another embodiment, a camera can monitor or track the test taker's eye movement, and the embodiment can determine that the test taker is not paying attention and/or is distracted. The testing process can then be paused until the test taker's attention is refocused.

The biometric data can be used to adapt the test based on the length of a test. For example, for tests of long duration, the biometric data may indicate an increase in anxiety and/or stress. In response, the testing process can be modified to allow for a short break before resuming the test.

Whatever biometric data are gathered, and whatever determination is made from that biometric data, the test and/or testing process can be adapted in some manner, thereby resulting in a tailored experience for the test taker. The testing process will then accurately gauge the knowledge of the test taker, rather than the test taker's ability to take the test.

The biometric data that are gathered from a test taker can also be used in other ways. For example, a test administrator can use these data to determine the effectiveness of a question or an entire test. This permits a test to be modified to be more effective for a broader audience. The test administrator can use the biometric data to determine that certain questions covered material with which the test takers were not comfortable. These data can be used to perhaps spend more time on that material in the future, revise or get rid of those questions, or some other action. Test administrators can use the biometric data to compare the success rate of different types of questions that test the same knowledge. This can enable instructors to better prepare students for different types of questions such as true/false, multiple choice and matching questions.

In another embodiment, the biometric data are used in a contrarian manner. For example, a certain test such as a driver's test or a bar exam may very well want to determine any areas in which the test taker is not proficient. In this scenario, the biometric data may indicate that a certain question or set of questions make a test taker nervous. In such a case, the test can adapt to ask more questions in that subject area to determine if the user simply does not have the knowledge in that area.

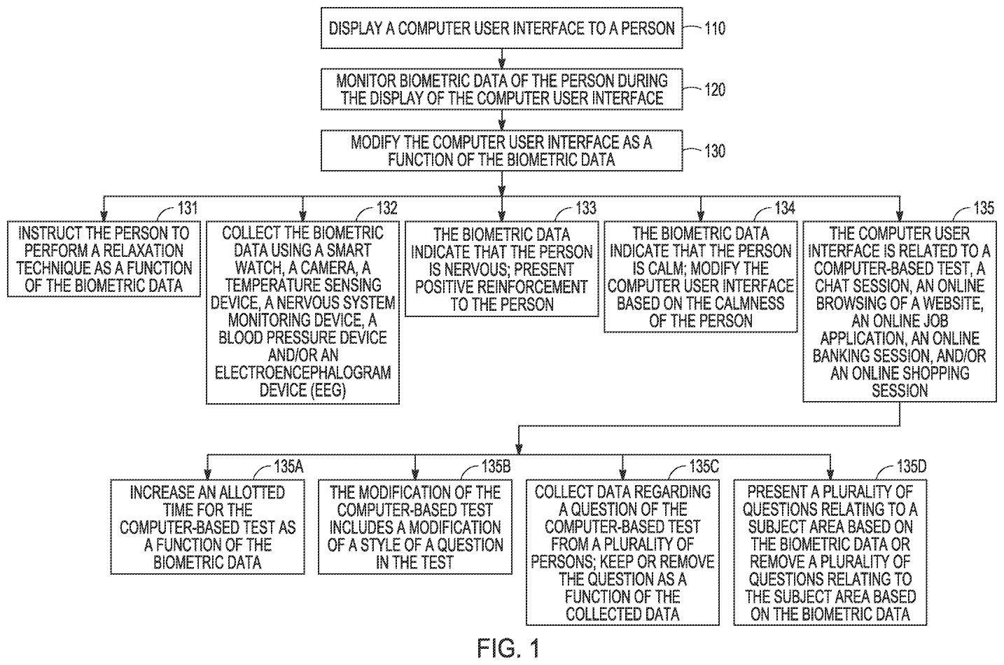

FIG. 1 is a block diagram illustrating example embodiments of operations and features of a system and method for generally modifying a computer-user interface as a function of biometric data gathered from the user of the computer interface, and more specifically modifying a computer-based test as a function of biometric data gathered from a test taker. FIG. 1 includes a number of process and feature blocks 110-135D. Though arranged substantially serially in the example of FIG. 1, other examples may reorder the blocks, omit one or more blocks, and/or execute two or more blocks in parallel using multiple processors or a single processor organized as two or more virtual machines or sub-processors.

Referring now specifically to FIG. 1, at 110, a computer user interface is displayed to a person. At 120, biometric data of the person are monitored during the display of the computer user interface. At 130, the computer user interface is modified as a function of the biometric data.

As noted at 131, the person is instructed to perform a relaxation technique as a function of the biometric data. For example, the person could be instructed to step back from the computer user interface and perform some breathing exercises.

At 132, the biometric data are collected using a smart watch, a camera, a temperature sensing device, a nervous system monitoring device, a blood pressure device and/or an electroencephalogram device (EEG). One of skill in the art will know which device to use for which purpose. For example, if the system would like to determine the nervous state of the person, a smart watch can be used to monitor the pulse of the person. As another example, if the system wanted to determine the attention level of the person, a camera can be used to determine if the person is looking at or away from the user interface.

At 133, the biometric data indicate that the person is nervous, and the system presents positive reinforcement to the person. For example, as noted above, in a test taking scenario, the system can inform the user that he or she has answered several questions correctly, and this could serve to decrease the nervousness of the person.

At 134, the biometric data indicate that the person is calm, and in response the system modifies the computer user interface based on the calmness of the person. For example, in the test taking situation, in view of the calmness of the person, the test can be made harder to challenge the person without concern of providing a harder test to a nervous person.

As noted at 135, the computer user interface can be related to a computer-based test, a chat session, an online browsing of a website, an online job application, an online banking session, and/or an online shopping session, just to name a few application examples.

At 135A, in connection with a computer-based test, the system increases an allotted time for the computer-based test as a function of the biometric data. For example, if the biometric data indicate that the test taker is nervous and the system recommends that the test taker take a break, the system can then compensate for that by increasing the test taking time (and informing the user of such an extension).

As indicated at 135B, the modification of the computer-based test includes a modification of a style of a question in the test. For example, as indicated above, if a question is rather long and includes a plurality of parts, the question can be broken up into shorter inquiries, such as several true/false questions.

At 135C, data are collected regarding a question of the computer-based test from a plurality of persons, and the system keeps or removes the question as a function of the collected data. For example, the question can be kept or removed based on an increase or decrease in the nervousness of the plurality of persons when presented with the question.

Similarly, at 135D, the system presents a plurality of questions relating to a subject area based on the biometric data or removing a plurality of questions relating to the subject area based on the biometric data. For example, in a state bar exam, if a test taker is nervous during questions relating to real property, more questions relating to real property can be presented to the test taker to be sure that the person truly understands the subject matter. In contrast, during an introductory college course on history, the test can remove questions relating to European history when it is determined that the person answering those questions is nervous during those questions.

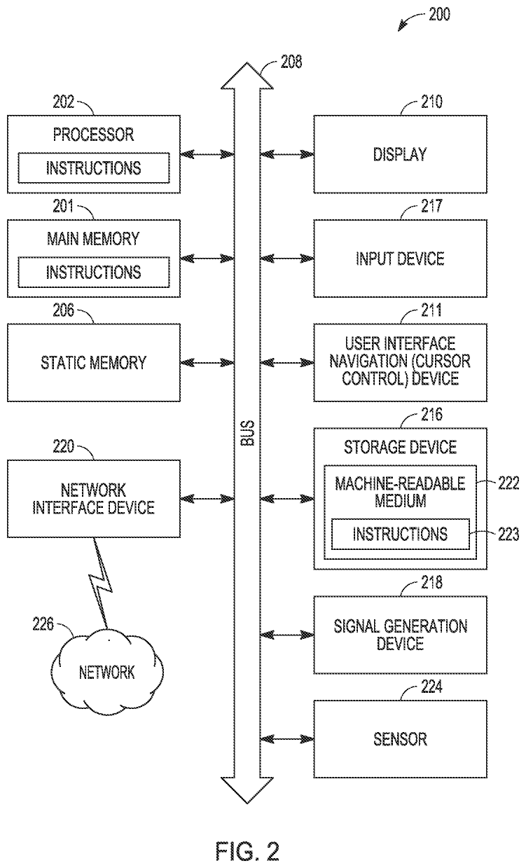

FIG. 2 is a block diagram illustrating a computing and communications platform 200 in the example form of a general-purpose machine on which some or all the operations of FIG. 1 may be carried out according to various embodiments. In certain embodiments, programming of the computing platform 200 according to one or more particular algorithms produces a special-purpose machine upon execution of that programming. In a networked deployment, the computing platform 200 may operate in the capacity of either a server or a client machine in server-client network environments, or it may act as a peer machine in peer-to-peer (or distributed) network environments.

Example computing platform 200 includes at least one processor 202 (e.g., a central processing unit (CPU), a graphics processing unit (GPU) or both, processor cores, compute nodes, etc.), a main memory 201 and a static memory 206, which communicate with each other via a link 208 (e.g., bus). The computing platform 200 may further include a video display unit 210, input devices 217 (e.g., a keyboard, camera, microphone), and a user interface (UI) navigation device 211 (e.g., mouse, touchscreen). The computing platform 200 may additionally include a storage device 216 (e.g., a drive unit), a signal generation device 218 (e.g., a speaker), a sensor 224, and a network interface device 220 coupled to a network 226.

The storage device 216 includes a non-transitory machine-readable medium 222 on which is stored one or more sets of data structures and instructions 223 (e.g., software) embodying or utilized by any one or more of the methodologies or functions described herein. The instructions 223 may also reside, completely or at least partially, within the main memory 201, static memory 206, and/or within the processor 202 during execution thereof by the computing platform 200, with the main memory 201, static memory 206, and the processor 202 also constituting machine-readable media.

While the machine-readable medium 222 is illustrated in an example embodiment to be a single medium, the term “machine-readable medium” may include a single medium or multiple media (e.g., a centralized or distributed database, and/or associated caches and servers) that store the one or more instructions 223. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, also contemplated are examples that include the elements shown or described. Moreover, also contemplated are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to suggest a numerical order for their objects.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

### EXAMPLES

Example No. 1 is a process for displaying a computer user interface to a person; monitoring biometric data of the person during the display of the computer user interface; and modifying the computer user interface as a function of the biometric data.

Example No. 2 includes all the features of Example No. 1, and optionally includes a process comprising instructing the person to perform a relaxation technique as a function of the biometric data.

Example No. 3 includes all the features of Example Nos. 1-2, and optionally includes a process comprising collecting the biometric data using one or more of a smart watch, a camera, a temperature sensing device, a nervous system monitoring device, a blood pressure device and an electroencephalogram device (EEG).

Example No. 4 includes all the features of Example Nos. 1-3, and optionally includes a process wherein the biometric data indicate that the person is nervous; and comprising presenting positive reinforcement to the person.

Example No. 5 includes all the features of Example Nos. 1-4, and optionally includes a process wherein the biometric data indicate that the person is calm; and comprising modifying the computer user interface based on the calmness of the person.

Example No. 6 includes all the features of Example Nos. 1-5, and optionally includes a process wherein the computer user interface comprises one or more of a computer-based test, a chat session, an online browsing of a website, an online job application, an online banking session, and an online shopping session.

Example No. 7 includes all the features of Example Nos. 1-6, and optionally includes a process comprising increasing an allotted time for the computer-based test as a function of the biometric data.

Example No. 8 includes all the features of Example Nos. 1-7, and optionally includes a process wherein the modifying of the computer-based test comprises modifying a style of a question in the test.

Example No. 9 includes all the features of Example Nos. 1-8, and optionally includes a process comprising collecting data regarding a question of the computer-based test from a plurality of persons, and keeping or removing the question as a function of the collected data.

Example No. 10 includes all the features of Example Nos. 1-9, and optionally includes a process comprising presenting a plurality of questions relating to a subject area based on the biometric data or removing a plurality of questions relating to the subject area based on the biometric data.

Example No. 11 is a machine-readable medium comprising instructions that when executed by a processor execute a process comprising displaying a computer user interface to a person; monitoring biometric data of the person during the display of the computer user interface; and modifying the computer user interface as a function of the biometric data.

Example No. 12 includes all the features of Example No. 11, and optionally includes a machine-readable medium comprising instructions for instructing the person to perform a relaxation technique as a function of the biometric data.

Example No. 13 includes all the features of Example Nos. 11-12, and optionally includes a machine-readable medium comprising instructions for collecting the biometric data using one or more of a smart watch, a camera, a temperature sensing device, a nervous system monitoring device, a blood pressure device and an electroencephalogram device (EEG).

Example No. 14 includes all the features of Example Nos. 11-13, and optionally includes a machine-readable medium wherein the biometric data indicate that the person is nervous; and comprising instructions for presenting positive reinforcement to the person.

Example No. 15 includes all the features of Example Nos. 11-14, and optionally includes a machine-readable medium wherein the computer user interface comprises one or more of a computer-based test, a chat session, an online browsing of a website, an online job application, an online banking session, and an online shopping session.

Example No. 16 includes all the features of Example Nos. 11-15, and optionally includes a machine-readable medium comprising instructions for increasing an allotted time for the computer-based test as a function of the biometric data.

Example No. 17 includes all the features of Example Nos. 11-16, and optionally includes a machine-readable medium wherein the modifying of the computer-based test comprises modifying a style of a question in the test.

Example No. 18 includes all the features of Example Nos. 11-17, and optionally includes a machine-readable medium comprising instructions for collecting data regarding a question of the computer-based test from a plurality of persons, and keeping or removing the question as a function of the collected data.

Example No. 19 includes all the features of Example Nos. 11-18, and optionally includes a machine-readable medium comprising instructions for presenting a plurality of questions relating to a subject area based on the biometric data or removing a plurality of questions relating to the subject area based on the biometric data.

Example No. 20 is a system comprising a computer processor; and a computer memory coupled to the computer processor; wherein the computer processor and computer memory are operable for displaying a computer user interface to a person; monitoring biometric data of the person during the display of the computer user interface; and modifying the computer user interface as a function of the biometric data.

1. A process comprising:
displaying a computer user interface to a person;
monitoring biometric data of the person during the display of the computer user interface; and
modifying the computer user interface as a function of the biometric data.

2. The process of claim 1, comprising instructing the person to perform a relaxation technique as a function of the biometric data.

3. The process of claim 1, comprising collecting the biometric data using one or more of a smart watch, a camera, a temperature sensing device, a nervous system monitoring device, a blood pressure device and an electroencephalogram device (EEG).

4. The process of claim 1, wherein the biometric data indicate that the person is nervous; and comprising presenting positive reinforcement to the person.

5. The process of claim 1, wherein the biometric data indicate that the person is calm; and comprising modifying the computer user interface based on the calmness of the person.

6. The process of claim 1, wherein the computer user interface comprises one or more of a computer-based test, a chat session, an online browsing of a website, an online job application, an online banking session, and an online shopping session.

7. The process of claim 6, comprising increasing an allotted time for the computer-based test as a function of the biometric data.

8. The process of claim 6, wherein the modifying of the computer-based test comprises modifying a style of a question in the test.

9. The process of claim 6, comprising collecting data regarding a question of the computer-based test from a plurality of persons, and keeping or removing the question as a function of the collected data.

10. The process of claim 6, comprising presenting a plurality of questions relating to a subject area based on the biometric data or removing a plurality of questions relating to the subject area based on the biometric data.

11. A non-transitory machine-readable medium comprising instructions that when executed by a processor execute a process comprising:
displaying a computer user interface to a person;
monitoring biometric data of the person during the display of the computer user interface; and
modifying the computer user interface as a function of the biometric data.

12. The non-transitory machine-readable medium of claim 11, comprising instructions for instructing the person to perform a relaxation technique as a function of the biometric data.

13. The non-transitory machine-readable medium of claim 11, comprising instructions for collecting the biometric data using one or more of a smart watch, a camera, a temperature sensing device, a nervous system monitoring device, a blood pressure device and an electroencephalogram device (EEG).

14. The non-transitory machine-readable medium of claim 11, wherein the biometric data indicate that the person is nervous; and comprising instructions for presenting positive reinforcement to the person.

15. The non-transitory machine-readable medium of claim 11, wherein the computer user interface comprises one or more of a computer-based test, a chat session, an online browsing of a website, an online job application, an online banking session, and an online shopping session.

16. The non-transitory machine-readable medium of claim 15, comprising instructions for increasing an allotted time for the computer-based test as a function of the biometric data.

17. The non-transitory machine-readable medium of claim 15, wherein the modifying of the computer-based test comprises modifying a style of a question in the test.

18. The non-transitory machine-readable medium of claim 15, comprising instructions for collecting data regarding a question of the computer-based test from a plurality of persons, and keeping or removing the question as a function of the collected data.

19. The non-transitory machine-readable medium of claim 15, comprising instructions for presenting a plurality of questions relating to a subject area based on the biometric data or removing a plurality of questions relating to the subject area based on the biometric data.

20. A system comprising:
a computer processor; and
a computer memory coupled to the computer processor;
wherein the computer processor and computer memory are operable for:
displaying a computer user interface to a person;
monitoring biometric data of the person during the display of the computer user interface; and
modifying the computer user interface as a function of the biometric data.
